(19) United States
(12) Patent Application Publication
(10) Pub. No.: US 2012/0252795 A1
(43) Pub. Date: Oct. 4, 2012

(54) ORAL LYSOPHILISATES CONTAINING PVP/VA

(75) Inventor: Thanh-Tam Nguyen, Limeil-Brevannes (FR)

(73) Assignee: CEPHALON FRANCE, Maisons-Affort Cedex (FR)

(21) Appl. No.: 13/487,421

(22) Filed: Jun. 4, 2012

(30) Foreign Application Priority Data
Jan. 18, 2010 (FR) 1050290

(51) Int. Cl. A61K 47/32; A61K 31/337; A61P 29/00; A61K 31/216; A61P 9/00; A61P 3/06; A61K 31/5415; A61K 31/4422

(52) U.S. Cl. 514/226.5; 514/772.5; 514/449; 514/347; 514/543

(57) ABSTRACT

The present invention relates to novel oral pharmaceutical compositions in lyophilized form, in which the dissolution and the bioavailability of the active ingredient that they contain are improved. The compositions according to the invention comprise in particular a polyvinyl acetate/polyvinylpyrrolidone copolymer. The oral lyophilisates according to the invention are particularly suitable for the production of medicaments based on active ingredients which have low solubility or very low solubility in water or which are virtually insoluble in water.

### CROSS REFERENCE TO RELATED APPLICATIONS

This application claims the benefit of the filing date of PCT Patent Application No. PCT/EP2011/050581, filed Jan. 18, 2011, now published as WO/2011/086194, publication date Jul. 21, 2011, which claims the benefit of the filing date of European Patent Application No. FR1050290, filed Jan. 18, 2010, and the benefit of the filing dated of U.S. Provisional Patent Application No. 61/312,048, filed Mar. 9, 2010, the disclosures of which are hereby incorporated herein by reference.

### TECHNICAL FIELD

The present invention relates to novel oral pharmaceutical compositions in lyophilized form, in which the dissolution and the bioavailability of the active ingredient that they contain are improved. The compositions according to the invention comprise in particular a polyvinyl acetate/polyvinylpyrrolidone copolymer. The oral lyophilisates according to the invention are particularly suitable for the production of medicaments based on active ingredients which have low solubility or very low solubility or which are virtually insoluble in water.

### DEFINITIONS

Oral Lyophilisate

In the present invention, the term “oral lyophilisate” will denote a pharmaceutical composition which is lyophilized (i.e. which has undergone successive steps of freezing, drying and sublimation under reduced pressure) and which is intended for oral administration. These oral lyophilisates are most commonly in the form of dry and porous tablets obtained by lyophilization, which disintegrate very rapidly as soon as they are brought into contact with saliva. These tablets may contain one or more pharmaceutical active ingredients.

Spray-Drying

The term “spray-drying” will be used in the present application to describe the process of dehydration which makes it possible to go from a liquid form (solution, suspension) to a dry form (powder) by pulverization. This process consists in pulverizing a solution or a suspension in the form of droplets or of fine particles in an environment which allows rapid drying thereof through evaporation of the solvent under the effect of heat optionally combined with a stream of air or of another gas.

Bioavailability and Absorption

The term “absorption” denotes the passing of an active ingredient from the external medium (saliva, gastrointestinal fluid) to the bloodstream.

In the present invention, the term “bioavailability” will be used to describe the fraction of active ingredient that is actually absorbed by the organism and reaches the bloodstream, relative to the dose of medicament contained in the pharmaceutical composition administered.

Copovidone/Copolyvidone

In the present invention, the terms “copovidone or copolyvidone” will be used to denote the vinyl acetate/polyvinylpyrrolidone copolymer.

This water-soluble copolymer is sold in particular under the trade names Kollidon VA®, Plasdone® or else Luviskol®. The particular grade sold under the name Kollidon VA 64 is obtained by polymerization of 6 parts of polyvinylpyrrolidone with 4 parts of vinyl acetate. This copolymer is listed as a pharmaceutical excipient in the European pharmacopeia and the Japanese pharmacopeia.

Dissolution and Solubilization

In the present invention, the term “dissolution” is used to describe the changing from the solid state to the state dissolved in water of a medicament, and more particularly of the active ingredient(s) that it contains; the dissolution can be quantified by quantitative determination (dissolution test). In the invention, the term “dissolution” will be used as a synonym of the term “solubilization” which corresponds to the visible change of a solid to the dissolved state (absence of particles in the solution).

Low Solubility

The terms “low solubility” or “with low solubility” or “with low water-solubility” denote, in the present invention, all active ingredients of which the solubility in water is defined as low to zero by the United States pharmacopeia (USP 32) according to the amount of water necessary for the dissolution of one part of solute:
Low solubility: 100 to 1000 parts of water necessary for dissolution of one part of solute.
Very low solubility: 1000 to 10 000 parts of water necessary.
Virtually insoluble: more than 10 000 parts of water necessary.

Microemulsion

The term “microemulsion” will be used to denote an emulsion obtained by using at least two nonionic surfactants including at least one main surfactant and one second “cosurfactant”, acting in combination with said main surfactant. Such a microemulsion has great stability and the globules of the emulsion are very small in size: of the order of a few nanometers to a few micrometers.

Wettability

In the present invention, the term “wettability” is intended to mean the ability of a liquid to remain in contact with a solid when these two elements are brought together. The degree of wettability is the result of the cohesive forces exerted on the liquid which oppose the spreading of said liquid on the surface of the solid, and of the adhesive forces that are exerted from the solid onto the liquid, which promote spreading of the latter. The chemical composition of the liquid and the chemical nature of the solid therefore influence the wettability. In the subsequent text, the term “wettability” will be used for active ingredients which have low solubility or very low solubility when they are brought into contact with the aqueous liquid phase necessary for the preparation of the oral lyophilisates which are subjects of the invention.

Conventional Surfactants

In the context of the present invention, the term “conventional surfactants” is intended to mean surfactants commonly used in the food and cosmetics industries for example, but which have not been approved for use in the pharmaceutical industry, and also the ionic surfactants approved for pharmaceutical use.

The present invention relates to the production of novel lyophilized pharmaceutical forms for oral administration of active ingredients. The oral lyophilisates according to the invention are capable of improving both the dissolution and the absorption in the digestive tract of low-solubility or even very-low-solubility active ingredients.

The lyophilized pharmaceutical forms (or oral lyophilisates) have a certain number of advantages compared with other oral galenical forms, such as the conventional tablets obtained by compression or the microgranules obtained by extrusion or granulation. This is because these solid forms are simultaneously stable, light and porous, which allows them to disintegrate rapidly in the buccal cavity on contact with saliva and thus allows rapid release of the active ingredient that they contain.

Rapid disintegration in the mouth allows, moreover, easy oral administration, which can be carried out without water and is thus particularly suitable for patients who encounter difficulties with oral absorption of solid forms, in particular young children and the elderly. The oral lyophilisates thus make it possible to improve treatment adherence.

In addition, lyophilization is known to confer a certain number of advantages, in particular in terms of preservation of the initial characteristics of the pharmaceutical active ingredient and an improvement in chemical stability of the latter (for example by avoiding hydrolysis or oxidation reactions). The lyophilized form also makes it possible to increase the physical stability of the active ingredient by preserving some of its characteristics during the production process (for example, the micronized state, the organoleptic properties, the crystallinity, and the surface treatment of the particles). Furthermore, the porous structure of the lyophilisates makes it possible to prevent a reaggregation of the particles of active ingredient at the time of disintegration of the tablet in water.

Lyophilized forms are thus advantageous since they can be formulated with large amounts of active ingredient and are suitable for virtually all types of therapeutic agents (excluding active ingredients such as proteins, which are sensitive to fluctuations in temperature), irrespective of their physicochemical properties. In particular, the lyophilized form is particularly suitable for the pharmaceutical formulation of active ingredients which have low solubility or very low solubility or which are even virtually insoluble in water.

In addition, they allow the production of tablets which contain a high dose of active ingredient, and of tablets containing one or more different active ingredients. Finally, these forms allow easy combination, within the same galenical unit (tablet), of one or more active ingredients.

The formulation of active ingredients with very low solubility, in oral lyophilisates, even if the low-solubility or insoluble active agent can be dispersed in the aqueous phase before the sublimation step, does not however make it possible to improve the subsequent solubility of this active agent in the digestive tract and in the buccal cavity in particular.

Similarly, the bioavailability of this or these active ingredient(s), which is in general correlated with the water-solubility thereof (i.e. an active agent with low water-solubility will in general have a low bioavailability in the organism), remains a problem that the existing formulations of oral lyophilisates do not make it possible to solve.

This is because lyophilized forms are obtained from a necessarily aqueous liquid phase comprising the active ingredient, in solid form or dissolved form (solution, suspension, dispersion or emulsion), since it is the sublimation of water (direct passage from the solid state to the gaseous state) which creates the particular porous structure desired. As a result, as soon as it is envisioned to introduce active ingredients with very low water solubility into lyophilized forms, the simplest solution for improving the dissolution thereof (and therefore the bioavailability thereof) in the organism is to use surfactants in the liquid phase. These surfactants, by associating on the molecular scale with the molecules of active ingredient during the sublimation phase, make it possible to improve the water solubility of said molecules.

However, the conventional surfactants are often not recommended or even incompatible for pharmaceutical formulations owing to the possible side effects that they induce (irritations in particular). Furthermore, the tolerance of said conventional surfactants by the organism is often low and decreases with the dose used.

Another means of increasing the solubility of active ingredients with low water solubility involves the use of organic solvents in which the active ingredient with low water solubility is dissolved. Evaporation of the organic solvent makes it possible to closely associate the active ingredient with the excipients present in the formulation. However, here again, these solvents are poorly tolerated by the organism, are expensive, are sometimes dangerous to handle since they are flammable and/or explosive and are harmful to the environment. The general problem addressed by the present invention is therefore that of improving the dissolution and the bioavailability of active ingredients which have low solubility or very low solubility or which are even virtually insoluble in water, in lyophilized oral formulations, without the addition of nonpharmaceutical and/or ionic conventional surfactants or organic solvents, which have a certain toxicity and/or are responsible for side effects that are harmful to the organism.

The lyophilized formulations are pharmaceutical forms described in particular in patents U.S. Pat. No. 3,855,712; U.S. Pat. No. 4,616,047; U.S. Pat. No. 4,490,407 and U.S. Pat. No. 5,843,347. These formulations may be based on a liquid phase in the form of a solution, a suspension or an emulsion, the water of which is subsequently sublimated, and may contain various excipients such as diluents, flavors or sweeteners, for example. These formulations have the advantage of completely and very rapidly disintegrating on contact with saliva or the aqueous medium.

Polyvinylpyrrolidone copolymers of grades having the following PVP/VA ratios: 30/70; 50/50; 70/30 and 20/80 are used in certain pharmaceutical products, such as granulating agents, spray-on bandages, or antiseptic, anesthetic or else antibiotic sprays, but they are soluble only in ethanol or isopropanol. They are water-dispersible but are not water-soluble.

Copovidone or polyvinylpyrrolidone copolymer having a PVP/VA ratio of 60/40 (6 parts of vinylpyrrolidone for 4 parts of vinyl acetate) is an excipient which is soluble in water, in isopropanol and in ethanol. The copovidone sold under the trademark Kollidon® VA 64 is referenced for having several applications in pharmacy. It is first and foremost an excellent binder for tablets or microgranules obtained by dry or wet granulation.

It can also be used for tablets obtained by direct compression (without prior granulation) owing to its plasticity and to the shape and distribution of its particles. Copovidone is also used for the production of coating or undercoating films having good elasticity for granules or tablets. Similarly, copovidione can be used in the composition of topical sprays. Finally, this copolymer is also referenced as a retarding matrix agent, in combination with other excipients, in the production of controlled-release formulations. For the latter application, it is mentioned that copovidone can be used in lyophilized form.

In the Yamanouchi document (U.S. Pat. No. 6,465,009), copovidone is cited in nonlyophilized formulations as a possible derivative of polyvinylpyrrolidone (PVP) that can be used as a compression agent for the production of fast-disintegrating oral tablets having a rapid disintegration speed.

Polyvinylpyrrolidone/vinyl acetate copolymers and copovidone in particular, have already been described in lyophilized formulations. Thus, in U.S. Pat. No. 3,960,476, this copolymer is used as a filler in the composition of hair dye in powder form, obtained by lyophilization or spray-drying. In these nonpharmaceutical formulations, the copovidone is part of the composition of the resin forming the film of the dyes described in this document.

Polyvinylpyrrolidone/vinyl acetate copolymers have already been the subject of use as excipients in oral pharmaceutical formulations.

Thus, BASF patent EP0545209, which relates to the production of redispersible powders of polyvinylpyrrolidone/vinyl acetate copolymers that are insoluble in water (of which the content by weight of vinyl acetate is greater than 50%), also describes oral formulations in the form of matrix tablets in which such polymers are used. These polymers pre-reduced in powder form by spray-drying are redispersed in an aqueous phase and then used as coating agents for controlling the speed of release of the active ingredient, by means of alternating coating layers comprising different vinylpyrrolidone/vinyl acetate ratios.

The Bayer document U.S. Pat. No. 5,707,655 describes the preparation of solid solutions based on low-solubility active ingredients, making it possible to improve the stability of these active agents. These formulations are obtained by conventionally formulating the active agents of interest with polymeric auxiliary agents, among which is copovidone, and then, in a second step, by heating the formulation thus prepared so as to melt the active agent of interest and the excipients together (thermal dissolution). The copovidone is in this case preferentially used as an agent for improving the stability of the active agent and for delaying the release thereof. Owing to the heat treatment, only active agents resistant to high temperatures can be used in these formulations.

The activity of vinylpyrrolidone/vinyl acetate copolymers as stabilizers of enzymatic activity in liquid formulations in which the liquid phase is subsequently evaporated by spray-drying is also known. Thus, in document U.S. Pat. No. 3,860,484, a vinylpyrrolidone/vinyl acetate copolymer containing 50 mol percent of vinyl acetate (i.e. having a PVP/VA ratio of 50/50) is used for the stabilization of enzymes with which it is brought into contact in an aqueous solution. The water is subsequently sublimated under vacuum and the enzymes contained in the dry formulation obtained exhibit a stabilized biological activity. The vinylpyrrolidone/vinyl acetate copolymers are in this case used as protective agents for reducing the risk of conformational changes, of inactivation or other denaturation that the enzymes may undergo before their use.

This protective agent activity of copovidone, but this time for the protection of a chemical agent very sensitive to variations in pH (omeprazole), is also described in US Patent Publication Application No. 2002/0128293 in which it is used as a stabilizer and as a diluent for omeprazole, by simple combination with the latter in a granule.

In the article titled “Solid Dispersions of Isopropylantipyrin”, Bogdanova et al. (1984), show that coprecipitates of a highly insoluble active ingredient (isopropylantipyrin) and of copovidone obtained after evaporation of an organic solvent (chloroform) do not improve the rate of dissolution of this active agent.

In the article titled “Characterization and Dissolution Study of Solid Dispersion with PVP/VA Copolymers” (STP Pharma Sciences, 2, 186-192, 1992), Zingone et al. describe formulations of insoluble active ingredient (indomethacin) in the form of solid dispersions obtained by evaporation, under vacuum, of an organic solvent (ethanol) in which the active ingredient and copovidone have been previously dissolved. The resulting coprecipitate of active ingredient and copovidone has the particularity of improving the dissolution of indomethacin at acidic pH, and this increase is proportional to the copovidone/active agent ratio. On the other hand, at neutral pH (7.4), the presence of copovidone does not increase the solubility of the active ingredient; on the contrary, the higher the proportion of copovidone, the lower the rate of dissolution of the indomethacin.

As has been demonstrated above, the current lyophilized formulations do not make it possible to significantly improve the rate of dissolution and the bioavailability of pharmaceutical active ingredients with low water solubility or which are virtually insoluble.

Moreover, the existing formulations in coprecipitate forms which make it possible to improve the rate of dissolution of these active agents are not satisfactory from both a pharmaceutical and an industrial point of view since they necessarily involve the use of organic solvents which, in addition to the precautions for use that they require on the industrial scale, are toxic to the human organism and the environment.

The same is true for the formulations based on conventional surfactants, which are compounds that are poorly tolerated by the organism and the use of which is not recommended for the production of pharmaceutical formulations.

There is therefore an unsatisfied need for oral lyophilized formulations which make it possible to improve the rate of dissolution of active ingredients with low water solubility and for which the production process does not involve any organic solvent or any conventional surfactant.

The present invention advantageously solves this problem since the inventors have shown that the use of copovidone in lyophilized formulations obtained from a liquid aqueous phase free of organic solvent and of ionic surfactant makes it possible to significantly improve both the rate of dissolution and the bioavailability of active agents which have low solubility or very low solubility or which are virtually insoluble in water.

In addition, the oral lyophilisates according to the invention have the advantage of being pH-independent insofar as the polyvinylpyrrolidone/vinyl acetate copolymer is nonionic. Finally, the lyophilized formulations according to the invention have the advantage of being able to be used for the formulation of active agents exhibiting very low concentrations.

The present invention relates to pharmaceutical compositions in oral lyophilisate forms, intended to improve the wettability, the hydrophilization and the solubilization of low-solubility active ingredients with the aim of improving the bioavailability thereof.

The present invention also relates to the process for producing these pharmaceutical forms.

A feature of the pharmaceutical formulations in accordance with the invention is that they are produced from a liquid aqueous phase free of organic solvent and of ionic surfactant, comprising at least one polyvinyl-pyrrolidone/polyvinyl acetate copolymer, the aqueous phase being subjected to a subsequent lyophilization step. Said polyvinylpyrrolidone/polyvinyl acetate copolymer has a PVP/VA molar ratio of 60/40.

The oral lyophilisates in accordance with the invention are obtained after sublimation of the water contained in a liquid aqueous phase free of organic solvent and of ionic surfactant, comprising at least one polyvinyl-pyrrolidone/polyvinyl acetate (PVP/VA) copolymer having a PVP/VA ratio of 60/40. This copolymer is obtained by polymerization of units of vinylpyrrolidone (PVP) and units of vinyl acetate (VA).

In the present invention, this copolymer has a PVP/VA ratio of 60/40 which gives it a water-solubility that allows it to be distributed homogeneously around the active ingredient during the formation of the aqueous liquid phase, whether the active ingredient is dissolved in the form of particles (suspension, dispersion) or else dissolved in a lipid solvent present in the aqueous phase in the form of microdroplets (emulsion).

This copolymer, which results from polymerization of 6 parts of vinylpyrrolidone with 4 parts of vinyl acetate, is sold, for example, under the grade Kollidon® VA 64 (BASF) or under the grade Plasdone® S630 (ISP).

The PVP/VA copolymer of the invention advantageously represents between 0.1% and 80% by weight relative to the dry weight of the oral lyophilisate in accordance with the invention. Preferentially, the proportion of copovidone is between 1% and 65% by weight relative to the total dry weight of the oral lyophilisate, even more preferentially between 5% and 40% by weight.

The proportion of PVP/VA copolymer in the liquid aqueous phase of the formulations of the invention advantageously represents between 5% and 20% by weight relative to the total weight of the liquid phase.

The oral lyophilized tablets in accordance with the invention may contain one or more active ingredients which represent(s), as appropriate, between 0.1% and 60% by weight relative to the total weight of the lyophilisates. Preferentially, the proportion of the active ingredient(s) in the lyophilized tablets of the invention is between 1% and 40%.

Thus, the active ingredient/copovidone proportion of the lyophilisates of the invention ranges between 1:800 and 600:1. Preferentially, the active ingredient/copovidone proportion is between 1:40 and 8:1, even more preferentially between 1:20 and 5:1.

The lyophilized formulations in accordance with the invention can advantageously contain a very large number of active ingredients, or even mixtures of active ingredients, as has been described above. This is because the production process works with virtually all active ingredients, with the exception of those of which the activity is sensitive to variations in temperature (in particular enzymes). Specifically, the physicochemical behavior of the active ingredient, and in particular its solubility in water, is not an obstacle to the preparation of the oral lyophilisates according to the invention since the aqueous phase which serves to bring the active ingredient into close contact with the copolymer can without distinction be in the form of a solution, a suspension, a dispersion or else an emulsion or microemulsion. The step of sublimation of the water contained in the aqueous phase is not in fact limited by the presence of dispersed solid particles (suspension) or even of lipid microdroplets (emulsion) in which the active agent is in dissolved form.

The present invention is particularly suitable for the administration of active ingredients which have low solubility or very low solubility or even which are virtually insoluble in water. As a result, active ingredients of which the water-solubility will be described as low to very low, or even active ingredients termed insoluble, may advantageously be used.

By way of example, it is possible to envision using, in the present invention, the following low-solubility active ingredients: fenofibrate, meloxicam, products of the family of dihydropyridines (DHP) and salts thereof, for example methanesulfonate salts of dihydropyridine and hydrochlorides of dihydropyridine, but also theophylline, indomethacin, carbamazepine, azacyclonol, buclizine; diazepam, chlordiazepoxide, oxazepam, oxanamide, hydroxyphenamate, phenaglycodol, haloperidol, perphenazine, thiothixene, mebutamate; diphenylhydantoin, metharbital, methsuximide, paramethadione, phensuximide, primidone, trimethadione, acetaminophen (paracetamol), acetylsalicylic acid, theophylline, nitrofurantoin, idoxuridine; ampicillin, cefaclor, cephalexin, cefsulodin, cefotiam or else molecules belonging to the dihydropyridine group with in particular nifedipine and nicardipine, amlodipine, felodipine and nimodipine.

The present invention can also be used for active agents that are more soluble, such as modafinil, armodafinil, fentanyl, bendamustine, cyclobenzaprine, arsenic trioxide, lestaurtinib, amphotericin B, carbinoxamine, sulpiride, diltiazem, diazepam, apomorphine, fluoxetine, capsaicin, nadoxolol, nimlodipine, dopexamine, captopril, erythromycin, buflomedil, loperamide, tiagabine, metoclopramide, clarithromycin, adrafinil, selegiline, phloroglucinol, doxorubicin, norfloxacine, lisinopril, bexarotene, tizanidine, verapamil, nitrendipine or zonisamide.

The lyophilized formulations according to the invention may advantageously contain one or more binders or viscosity modifiers, or else gelling agents. These excipients are conventionally used to confer a satisfactory viscosity on the aqueous phase intended to be lyophilized, i.e. a viscosity which makes it possible both to have a homogeneous distribution of the liquid phase in the individual cavities and to maintain the various excipients and active agents in suspension in the liquid phase, and also to confer sufficient mechanical strength on the final products so that they can be handled during packaging and use.

The aim of these binders is to provide good homogeneity and good stability of the liquid phase which must have an acceptable flow so that it can be used on the industrial scale. Specifically, the binders make it possible to prevent sedimentation of the particles contained in the liquid phase and to provide the final lyophilized product with a correct texture, for example by conferring sufficient hardness on the tablet so that it can be easily removed from its blister without breaking, while at the same time retaining sufficient porosity to be able to degrade very rapidly as soon as it comes into contact with saliva.

Such binders comprise all the water-soluble or water-dispersible substances that allow cohesion of the mass of the tablet and are pharmaceutically acceptable and inert with regard to the active ingredient(s) of interest.

The binders are chosen in particular from polypeptides, such as gelatin, colloids, high-molecular-weight polysaccharides, large polymers capable of giving colloidal solutions, such as resins or natural gums (for example, gum Arabic, gum tragacanth) or semisynthetic gums (for example, xanthan gum or glycosylglucans), dextran, in particular the grades known as Dextran 20, 40 and 70, dextrin, alginates, in particular sodium alginate, pectinates, carboxymethylcellulose, water-dispersible starch derivatives, colloidal silicas or bentonites.

Generally, the binders of the present invention represent between approximately 0.01% and up to approximately 30% of the dry mass of the final oral lyophilisate, preferentially between approximately 0.5% and 20%.

The oral lyophilisates according to the invention may advantageously contain fillers or diluents, which are excipients commonly used in lyophilized formulations.

These agents are preferentially pharmaceutically acceptable water-soluble substances such as sugars and derivatives thereof, for instance glucose, lactose, glycine, maltodextrin, isomalt, or cyclodextrins and derivatives thereof, or alcohol sugars such as mannitol, sorbitol or xylitol, for example.

Preferably, mannitol is used as filler for implementing the invention, said mannitol being used alone or in combination with another filler, for instance dextran.

The diluents may also belong to the family of oxides, for instance magnesium oxide, carbonates (for instance calcium carbonate) or phosphates (such as tricalcium phosphate). The fillers may represent between 0.5% and 90% by weight relative to the total dry weight of the oral lyophilisates. The preferred amount of diluent is between approximately 10% and approximately 50% relative to the dry weight of the lyophilized tablets.

Optionally, the formulations according to the present invention also contain one or more nonionic surfactants approved for pharmaceutical use which make it possible to improve the wettability and therefore the rate of dissolution of the low-solubility or insoluble active ingredients.

Among the nonionic surfactants approved for pharmaceutical use, the following surfactants will in particular be preferred: polysorbates, poloxamers, sugar esters and fatty acid esters, for example PEG-palmitostearate, PEG-6 caprylic/capric glycerides, glyceryl stearate and sorbitan stearate.

Preferentially, as nonionic surfactant for preparing the formulations according to the invention, use will be made of polysorbates of which the chemical name is polyoxyethylene sorbitan monolaurate, which are derivatives of pegylated sorbitan. These surfactants are, for example, sold under the trademark Tween®.

Polysorbates exist as various grades from Polysorbate 20 up to Polysorbate 80. For the formulations according to the invention the Polysorbate 20, 40, 60 and 80 grades are the preferred grades.

Alternatively, as nonionic surfactants for preparing the formulations according to the invention, use may be made of molecules of the polyoxyethylene sorbitan monooleate class, such as those sold, for example, under the trademarks Montanox® or Montane® (sold by Seppic), such as: Montane 20 which corresponds to sorbitan laurate, Montane 40 which corresponds to sorbitan palmitate, Montane 60 which corresponds to sorbitan stearate, or else Montane 80 which corresponds to sorbitan oleate.

Preferentially, the amount of nonionic surfactant present in the lyophilized formulations according to the invention is between 0.01% and 20% by weight relative to the total dry weight of the lyophilisate, more preferentially between 0.05% and 0.5% for the solutions/suspensions and 1% to 15% for the emulsions or microemulsions.

When the active ingredient intended to be used in the oral lyophilisates in accordance with the invention is particularly insoluble in water, it may be advantageous to incorporate it in dissolved form in a lipophilic liquid or emulsifier.

The active ingredient/lipophilic liquid whole then forms an emulsion with the liquid aqueous phase prepared before the sublimation step.

Thus, where appropriate, the substances described hereinafter may be used as emulsifier, in particular mixtures of mono-, di- and triglycerides and of mono- and diesters of polyethylene glycol (PEG), for instance those available under the trademarks such as Labrafil® (oleoryl macrogol 6 glucosides), Labrafac® (medium-chain triglycerides, propylene glycol dicaprylocaprate), Labrasol® (caprylocaprylmacrogol 8 glycerides); medium-chain glycerides (i.e. glycerides containing from 6 to 12 carbon atoms), partial glycerides, i.e. fatty acid esters of glycerol in which only a portion of the hydroxyl groups is esterified; monoesters of glycerol, such as, in particular, glyceryl monostearate, or glyceryl monodicocoate; certain polyglycerides, such as palmitostearate (for example the product sold under the trademark Precirol® ATO5 from Gattefosse), polyglyceryl isostearate, polyglyceryl oleate or else polyglyceryl palmitostearate.

The oral lyophilisates according to the invention may also contain, either alone or in combination with one another, sweeteners, taste-masking agents or flavors intended to increase the palatability of the medicament in the mouth.

Such excipients are conventionally used to improve the taste of the active ingredient or at the very least to mask its unpleasant taste, as appropriate.

For example, the following sweeteners can be used in a standard manner in the formulations in accordance with the invention: sucrose, glucose, xylose, sucralose, acesulfame, saccharin, saccharinates, cyclamates, aspartame, ammonium glycyrrhizinate or else citric acid, ascorbic acid or tartaric acid.

Generally, any other substance normally used as a taste modifier in the pharmaceutical industry and which is compatible with the active ingredient(s) used can be used for preparing the pharmaceutical compositions according to the invention.

The amount of sweetener or of taste-masking agent is generally between 0.01% and approximately 5%, preferentially between approximately 0.05% and 1% by weight relative to the dry weight of the tablets according to the invention.

Colorants and preservatives may also be used in the formulations of the invention and are those normally used in pharmaceutical formulations in general and for coloring lyophilized oral tablets in particular.

They comprise, for example: amaranth, barley extract, caramel, cochineal, carotene, copper-chlorophyll complexes, iron oxides, riboflavin, grape skin extract, titanium dioxide, erythrosine or methylene blue, for example.

In addition to the excipients mentioned above, the lyophilized formulations according to the invention may also contain, conventionally, other additional “cohesion” excipients intended, for example, to prevent breaking of the tablets.

Among these excipients are in particular silica or hydrophilic diluents, for instance certain sugars, such as levulose for example.

The present invention also relates to the process for producing oral lyophilisates based on PVP/VA copolymer, which are capable of improving the dissolution of low-solubility active ingredients, comprising the following steps:

a. preparing a liquid aqueous phase free of organic solvent and of ionic surfactant, containing at least one pharmaceutical active ingredient, one filler and/or one binder and the PVP/VA copolymer of which the PVP/VA ratio is 60/40;

b. stirring the aqueous phase until a homogeneous mixture is obtained;

c. distributing the resulting homogeneous mixture into a preformed cavity (for example a mold or a blister);

d. freezing the liquid phase thus distributed at a temperature of approximately −20° C. to approximately −50° C. until a frozen mixture is obtained;

e. lyophilizing said frozen mixture; and

f. optionally, carrying out a second, drying step on the resulting lyophilized mixture.

The process for preparing the lyophilisates in accordance with the invention is based on a common lyophilization process.

In a first step, an aqueous liquid phase intended to be lyophilized and containing the active ingredient(s) of interest is prepared. In accordance with the present invention, this liquid phase does not contain organic solvents.

According to the physicochemical characteristics of the active ingredient(s) used and the initial forms, structures and particle sizes thereof, a solution, suspension, emulsion or a preparation precooled to a semi-frozen consistency of sorbet type can be advantageously prepared as liquid aqueous phase. This liquid phase therefore contains at least the active ingredient(s) of interest, a binder and/or filler, the PVP/VA copolymer and water.

Advantageously, other excipients can be added to this liquid aqueous phase free of organic solvent and of ionic surfactant, for instance, and in a nonlimiting manner: colorants, sweeteners, taste-masking agents or preservatives.

The mixing of the active ingredient and of the appropriate excipients including the copovidone is generally carried out in a mixer equipped with a vacuum system. When it is in solid form, the active ingredient and the excipients in powder form are mixed until an acceptable homogeneous mixture is obtained. If the active ingredient is in liquid form (for example dissolved in a lipid liquid), it is mixed in a similar manner with the excipients mentioned above, which can be in solid or liquid form without distinction.

Generally, this mixing step lasts 5 to 30 minutes under reduced pressure (generally from 100 to 300 HPa) with the aim of “degassing” the powders and allowing suctioning of the water.

The aqueous phase is then finished off by adding water to the previously formed solid or liquid mixture by suctioning. The resulting solution, suspension or emulsion is then mixed under reduced pressure (generally from 100 to 300 HPa) for a period of 30 to 90 minutes until a perfectly homogeneous mixture is obtained.

The amount of water introduced so as to form the aqueous phase intended to be lyophilized is determined in such a way that this phase has acceptable rheological properties, i.e. a viscosity which allows it to have good flow, to be able to be easily mixed to give a perfectly homogeneous phase and to be able to be easily divided up and distributed uniformly into individual molds or blisters. In most cases, the amount of water will be adjusted in such a way that the solid mass constitutes approximately between 30% and 80% of the mixture. In certain preparations, this proportion can vary between 40% and 60% relative to the dry mass of the final product.

In a second step, once completely homogenized, the liquid preparation obtained is distributed into the preformed cavities, generally in the form of thermoformed molds made of PVC, PVDC or aluminum foils.

The distribution step is carried out mechanically, the overall volume of liquid phase being divided up into unit doses having a predetermined shape, size and volume.

The amount of active ingredient(s) in the liquid phase and the shape and the size of the cavities are calculated so as to obtain a precisely defined amount of active ingredient(s) in each unit dose.

The cavities (for example the blisters) containing the product are then placed on the platforms of the lyophilizer. These platforms are metal supports for the blisters, which can be easily heated or cooled and which allow an abrupt modification of the temperature of the product contained in the blisters.

Once the cavities are filled with the suitable volume of liquid preparation, the freezing step begins. It starts at very low temperature (generally between −20° C. and −50° C.) below the eutectic point (i.e. the temperature at which there is total solidification of the liquid phase) at atmospheric pressure, for approximately 30 to 90 minutes, until a solid phase is obtained.

The conventional lyophilizers that can be used are in general mechanical lyophilizers using a compressor system, such as those sold, for example, by the companies Usifroid, Virtis or BOC Edwards.

At the end of this step, the molecules of active ingredient are immobilized, the therapeutic properties thereof remaining unimpaired insofar as the number of chemical reactions is virtually zero at these very low temperatures.

The temperature and pressure conditions of the freezing phase are adjusted according to the composition of the liquid phase which may, depending on the nature and the concentration of the filler and/or the binder and the copovidone, be more or less easy to freeze.

The preparation thus frozen is then subjected to sublimation, i.e. the product is dried under vacuum, passing from the solid phase to the gaseous phase in a controlled manner by applying thereto both heat and a low pressure (generally between 100 and 600 pbar), preferentially between approximately 150 and 400 pbar. The temperature of the platforms supporting the molds or blisters is taken rapidly from approximately −301-50° C. up to approximately +40/+70° C. while the temperature of the product increases gently from the freezing point up to +30/+50° C.

According to the amount of material contained in each mold, the sublimation phase lasts, in total, between 300 and 800 minutes.

Under these conditions, the ice is converted directly to vapor phase. The product resulting therefrom is a pharmaceutical formulation in the form of a porous solid which has retained its initial shape and the therapeutic qualities of the active ingredient(s).

Optionally, an additional drying phase (also called secondary drying) is applied to the product and the residual water (termed “bound” water) is extracted by desorption (i.e. by evaporation of the liquid phase into the gaseous phase, contrary to sublimation, which corresponds to the evaporation of a solid phase into an gaseous phase).

This is because the presence of water in the product is often responsible for rapid degradation of the active ingredient, resulting in poor storage stability of the product.

This secondary drying step is generally carried out at between 40° C. and 50° C. and at a pressure of approximately 100 pbar for a period of from 60 to 120 minutes. The average duration of secondary drying in the lyophilizer is between 60 and 90 minutes, but can advantageously be prolonged, without any negative impact on the product.

The final lyophilized tablets are then heat-sealed in their cavities, for example, in the case of blisters with aluminum foils at a temperature of between 120° C. and 160° C. for approximately 1 to 3 seconds.

The oral lyophilized formulations of the invention can thus be used for the production of medicaments comprising at least one active ingredient and in particular active ingredients which have low solubility or very low solubility or which are virtually insoluble in water.

### FIGURES

FIG. 1:

Dissolution curves for three lyophilized formulations in accordance with the invention (Formulations 4, 5 and 6) tested against a prior art formulation of Lipanthyl® 67 (Formulation 7). These formulations have the following compositions:

Formulation 4 (Batch 340.07): (50 mg copovidone)

Formulation 5 (Batch 342.07): (50 mg copovidone+5 mg polysorbate 80)

Formulation 6 (Batch 341.07): (100 mg copovidone+5 mg polysorbate 80)

Formulation 7: (Lipanthyl® 67): Fenofibrate comicronized with sodium lauryl sulfate (other excipients: lactose, pregelatinized starch, crospovidone, sodium stearate).

FIG. 2:

Comparative dissolution curves for oral lyophilized formulations of meloxicam in accordance with the invention: Formulations 15 (batch 125.07), 17 (batch 126.07) and 19 (Batch 138.07) with a commercial formulation available on the market (Mobic®).

### EXAMPLES

### Example 1

### Fenofibrate-Based Oral Lyophilisates

In this example, the preliminary intention was to compare the rates of dissolution of fenofibrate between a lyophilized formulation according to the invention and a prior art formulation existing on the market (Secalip®). Fenofibrate is an active ingredient intended for limiting hyperlipidemia, which has very low solubility in water (of the order of 0.25 mg/ml at 25° C.).

The oral lyophilisates in accordance with the invention are prepared in the following way: the copovidone is dissolved, with stirring, with the binder (Dextran 70) and the viscosity modifier (xanthan gum) in purified water at ambient temperature.

The fenofibrate in the form of micronized particles (i.e. particles having a size of less than approximately 20 μm) or nonmicronized particles is then incorporated into this solution, with stirring, until all the particles are wetted with the polymer solution. This verification is carried out visually: the unwetted particles remain at the surface of the water, the wetted particles themselves fall to the bottom.

The sweetener (aspartame) and the filler (mannitol) are then added to this suspension.

The proportions of each of the excipients are reproduced in Table 1.

The composition of the prior art formulation available on the market (Secalip® gelatin capsule, 100 mg) is the following: nonmicronized fenofibrate, lactose, pregelatinized starch, sodium lauryl sulfate, sodium stearate.

TABLE 1
Fenofibrate-based formulations according to the invention

| | Formulation No. | | |
|---|---|---|---|
| | 1 | 2 | 3 |
| Fenofibrate | 67.00 mg | 67.00 mg | 100.00 mg |
| | (micronized) | (micronized) | (nonmicronized) |
| Copovidone | 5.00 mg | 20.00 mg | 20.00 mg |
| Dextran 70 | 10.00 mg | 5.00 mg | 5.00 mg |
| Xanthan gum | 0.15 mg | 0.15 mg | 0.25 mg |
| Aspartame | 0.5 mg | 0.5 mg | 0.5 mg |
| Mannitol | 217.35 mg | 207.35 mg | 174.25 mg |
| Water* | 230.00 mg | 230.00 mg | 230.00 mg |

*the water is eliminated during the process

The stirring is maintained until the suspension obtained is completely homogeneous. Said suspension is then introduced into preformed blisters in such a way that the required amount of fenofibrate is incorporated into each unit dose. These blisters are in turn introduced into the lyophilizer (Usifroid® SMH90 model). The mixture is then frozen at a temperature of approximately −35° C. The water is then eliminated by sublimation by varying the temperature between −35° C. and +45° C. (for approximately 400 minutes) and the pressure between 400 microbar and 50 microbar, i.e. under conditions in which the ice is directly converted to water vapor.

The product resulting from this step is in the form of porous solid tablets which disintegrate rapidly (in less than 3 minutes) as soon as they are brought into contact with water.

Dissolution tests according to the United States pharmacopeia (USP 32, chapter No. 711) are then carried out on Formulation 3 of the invention (nonmicronized fenofibrate, 100 mg) and the Secalip® 100 mg.

The results of the dissolution tests are given in Table 2. These results show the superiority of the rate of dissolution of fenofibrate observed for the formulation according to the invention (Formulation 3) compared with the commercial formulation Secalip® having the same dosage.

TABLE 2

| | Percentage of dissolved fenofibrate | |
|---|---|---|
| | After 30 minutes | After 120 minutes |
| Oral lyophilisate according to | 0.4% | 0.6% |
| the invention | | |
| (Formulation 3) | | |
| 100 mg of nonmicronized | | |
| fenofibrate | | |
| 20 mg of copovidone | | |
| (Kollidon ® VA 64) | | |
| 100 mg gelatin capsule of | 0% | 0.1% |
| nonmicronized fenofibrate | | |
| (Secalip ® 100) | | |

Thus, these preliminary tests show that the copovidone-based formulation in accordance with the invention (the fenofibrate/copovidone ratio of which is 5:1) makes it possible, for a product as insoluble as fenofibrate, to multiply six-fold the solubility of this active ingredient with respect to the commercial formulation Secalip®, which remains totally insoluble after 30 minutes.

### Example 2

### Oral Lyophilisates of Micronized Fenofibrate (67 mg)

In this example, the intention was to further improve the dissolution results obtained in Example 1, comparing the rates of dissolution of fenofibrate between three formulations of the invention optimized for this active ingredient comprising 67 mg of fenofibrate in micronized form, with a commercially available formulation comprising 67 mg of fenofibrate comicronized with an ionic surfactant, sodium lauryl sulfate (Lipanthyl®/Laboratoires Fournier). This comicronized formulation is described in detail in patent EP0330532 incorporated herein by way of reference.

According to this patent, the comicronization of the surfactant and the fenofibrate improves the rate of dissolution of the latter, and this increase is directly correlated to the bioavailability of this active ingredient, which increases with the rate of dissolution.

Three lyophilized formulations in accordance with the invention were prepared in the same way as that described in Example 1, and then the rate of dissolution thereof was subsequently tested against the patented Lipanthyl® 67 formulation (Formulation 7).

The compositions of these formulations in terms of copovidone and, optionally, in terms of nonionic surfactant (polysorbate 80) are described below.

Formulation 4: (50 mg copovidone)

Formulation 5: (50 mg copovidone+5 mg polysorbate 80)

Formulation 6: (100 mg copovidone+5 mg polysorbate 80)

Formulation 7: (Lipanthyl® 67): Fenofibrate comicronized with sodium lauryl sulfate (other excipients: lactose, pregelatinized starch, crospovidone, sodium stearate).

The detailed compositions of these various formulations are reproduced in Table 3 below.

TABLE 3

| | Formulation | | | |
|---|---|---|---|---|
| | 4 | 5 | 6 | 7 |
| | Batch number | | | |
| | 340.07 | 342.07 | 341.07 | Lipanthyl ® |
| | Composition | | | |
| | mg/unit | mg/unit | mg/unit | mg/unit |
| Fenofibrate | 67 | 67 | 67 | 67 |
| (micronized) | | | | |
| Copovidone (Kollidon ® | 50 | 50 | 100 | |
| VA 64) | | | | |
| Polysorbate 80 | 0 | 5 | 5 | SLS |
| Mannitol | 183 | 178 | 128 | |
| Purified water(*) | 300 | 300 | 300 | |
| Total weight of dry | 300 | 300 | 300 | |
| lyophilisate | | | | |
| Characterization | tablet | tablet | tablet | gelatin |
| | | | | capsule |
| Hardness (in newtons) | 13 N | 25 N | 20 N | n/a |
| Rate of disintegration | 18 | 15 | 30 | n/a |
| | seconds | seconds | seconds | |

SLS: Sodium lauryl sulfate
(*)The water is eliminated by sublimation and drying during the process
n/a: not applicable

The hardness of the lyophilized tablets is determined using a Schleuniger® durometer.

The rate of disintegration of the lyophilized tablets is determined according to the method described in the United States pharmacopeia USP 32 (paragraph No. 701).

Six tablets of each formulation are placed in a basket which is immersed in an immersion liquid maintained at 37° C., at a constant rate of between 28 and 32 cycles per minute, until complete disintegration of the six tablets. The term “disintegration” is intended to mean the moment when any residue remaining on the screen of the basket forms a soft mass which does not contain a manifestly firm core. The disintegration time measured is the time after which the six tablets are entirely disintegrated.

The rate of dissolution of the fenofibrate is determined conventionally in a paddle dissolutest as described in the United States pharmacopeia USP 32 (paragraph No. 711).

The rate of dissolution corresponds to the time necessary for complete dissolution of the tablets immersed in the liquid medium with stirring.

Each formulation is prepared in the same way as in Example 1 above. The results of these tests are reported in Table 4.

TABLE 4

| | Percentage of dissolved fenofibrate as a | | | |
|---|---|---|---|---|
| | function of time | | | |
| | 5 | 10 | 15 | 20 |
| | minutes | minutes | minutes | minutes |
| Formulation 4 | 41.7 | 72.4 | 88.3 | 95.3 |
| (50 mg copovidone) | | | | |
| Fenofibrate/copovidone ratio = | | | | |
| 1.34 | | | | |
| Formulation 5 | 51.0 | 81.9 | 95.2 | 98.9 |
| (50 mg copovidone + 5 mg | | | | |
| polysorbate) | | | | |
| Fenofibrate/copovidone ratio = | | | | |
| 1.34 | | | | |
| Formulation 6 | 55.0 | 84.3 | 94.2 | 98.5 |
| (100 mg copovidone + 5 mg | | | | |
| polysorbate) | | | | |
| Fenofibrate/copovidone ratio = | | | | |
| 0.67 | | | | |
| Formulation 7 | 17.7 | 63.0 | 85.1 | 94.8 |
| (Sodium lauryl sulfate | | | | |
| (Lipanthyl ®)) | | | | |

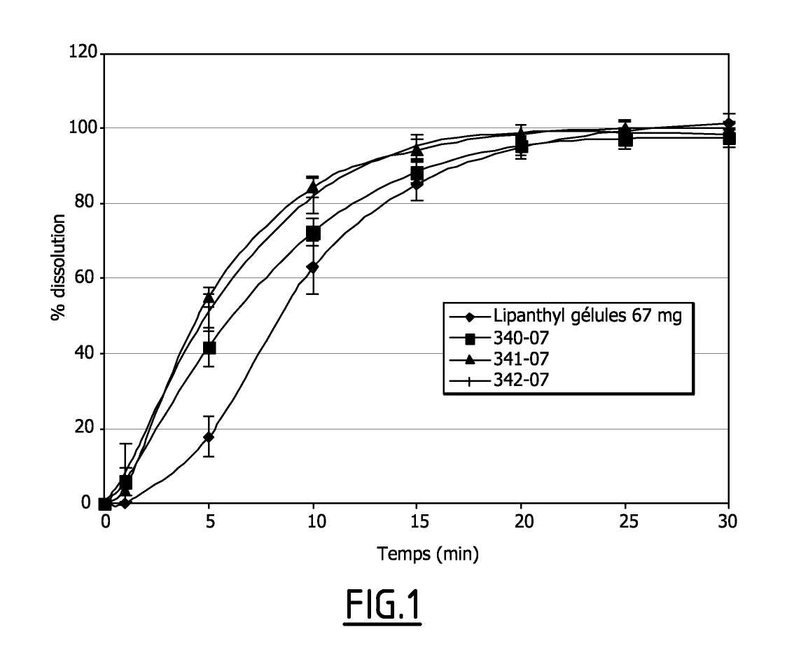

The results of the rates of dissolution are also reported in FIG. 1.

These results show that a formulation in accordance with the invention and free of surfactant (Formulation 4) has a better rate of dissolution (amount of fenofibrate dissolved as a function of time) than the prior art formulation in which the active agent is comicronized with an ionic surfactant.

The dissolution of the fenofibrate is in fact close to three times faster during the first 5 minutes for the three formulations of the invention compared with the commercial Lipanthyl® formulation.

Thus, approximately half the fenofibrate contained in the formulations of the invention is dissolved in 5 minutes.

Moreover, it appears that the formulations based on copovidone and surfactant are those which have the fastest rates of dissolution (more than 50% of fenofibrate dissolved in 5 minutes). It is also noted that the rate of dissolution increases with the amount of copovidone added (Formulation 6).

In the two examples which follow, two molecules which are in the process of being developed and which are particularly insoluble in water were tested in the formulations of the invention. These two molecules (referenced under CRL 42646 and CRL 42249C) are new chemical entities belonging to the dihydropyridine (DHP) group which exhibit therapeutic activity in the cardiovascular field.

### Example 3

### CRL 42646

The compound referenced under CRL 42646 is a molecule belonging to the dihydropyridine (DHP) methanesulfonate chemical class. This methanesulfonate salt is virtually insoluble in water, but has a solubility of 1% in orthophosphoric acid (0.2%) at pH=1.9 and a solubility in hydrochloric acid (0.01 N NCI) of 0.5%.

This substance has a tendency to precipitate in physiological fluids and has a very low gastrointestinal absorption.

With the aim of improving the stability, the solubility and therefore the absorption of this compound, three formulations based on CRL 42646 were prepared according to the process for producing the oral lyophilisates which is described in the previous examples, according to the steps of mixing of the powders, addition of water, homogenization, distribution, freezing and lyophilization.

These three formulations are the following:
A control formulation without copovidone (Formulation 8).
A formulation based on beta-cyclodextrin as stabilizer (Formulation 9).
A formulation according to the invention based on copovidone and polysorbate (Formulation 10).

The content of each of these formulations is given in detail in Table 5.

TABLE 5

| | Formulations | | |
|---|---|---|---|
| | 8 | 9 | 10 |
| | (mg) | (mg) | (mg) |
| CRL 42646 | 10 | 10 | 10 |
| Hydroxypropyl β-cyclodextrin | — | 62.5 | — |
| Kollidon ® VA 64 | — | — | 25 |
| Polysorbate 80 | — | — | 5 |
| Aerosil ® 200 | 5 | — | — |
| Mannitol | 250 | 200 | 200 |
| Water (eliminated after | 400 | 400 | 400 |
| lyophilization) | | | |
| Dissolution tests | | | |
| Percentage dissolved | 0% | 100% | 100% |
| Time necessary for dissolution | After 2 h | 5 min | 5 min |

This example illustrates first of all the fact that a “simple” formulation, i.e. a formulation free of solubilizing agent, does not make it possible, even after two hours, to dissolve the active ingredient CRL 42646.

Moreover, this example shows that it is possible, through the use of a complexing agent such as hydroxylpropyl β-cyclodextrin, to obtain a rapid dissolution of the active agent CRL 42646, but provided that a large amount of this agent is used (in the case in point, in an active ingredient/dissolution agent ratio of less than 1:6).

On the other hand, the composition according to the invention (Formulation 10) makes it possible to obtain complete dissolution of the active ingredient after 5 minutes without cyclodextrin but with an active ingredient/copovidone ratio of 0.4 and an active ingredient/dissolution agent (in this case the combination of copovidone and surfactant) ratio of 1:3. This means that approximately half as much dissolution agent is necessary in the formulation in accordance with the invention in order to obtain the same result as with the cyclodextrin-based formulation.

### Example 4

### CRL 42249C

The compound referenced under CRL 42249C is a molecule belonging to the dihydropyridine (DHP) hydrochloride chemical class. This hydrochloride is virtually insoluble in water (solubility<0.1 g/l), but is soluble in chloroform, methanol, ethanol and dimethyl sulfoxide (DMSO).

Dissolution tests were carried out on several formulations of CRL 42249C, in a manner identical to what was carried out in Example 3.

Several preliminary solubilization tests were carried out in an attempt to solubilize the active ingredient in various media in order to select the best solubilizing agents suitable for this active ingredient.

The solubilization test consists in preparing, beforehand, a 15 ml aqueous solution containing 0.5% to 5% of solubilizing agent from among the products of the list below. 200 mg of the CRL 42249C compound are then added and the mixture is left to stir for 12 h. The mixture is then filtered and the amount of active ingredient contained in the solution was determined by quantitative measurement. The following agents were thus used as solubilizing agents: citric acid, sorbic acid, polyvinylpyrrolidone (Povidone® 12), Poloxamer 407 (block polymer of polyoxyethylene and polyoxypropylene, sold under the trade name Pluronic® F127), Cremophor® EL (commercial mixture consisting of 35 mol of PEG per mole of castor oil and polyethylene glycol (PEG 300)). It was not possible to dissolve the CRL 42249C active agent for any of these formulations.

On the other hand, two lyophilized formulations in accordance with the invention, produced based on copovidone with or without nonionic surfactant, and also two formulations based on β-cyclodextrin, made it possible to dissolve this active ingredient.

The details of each of these formulations are given in Table 6.

TABLE 6

| | Formulations | | | |
|---|---|---|---|---|
| | 11 | 12 | 13 | 14 |
| | (mg) | (mg) | (mg) | (mg) |
| CRL 42249C | 200 | 200 | 200 | 200 |
| Hydroxypropyl β-cyclodextrin | 1250 | 1000 | — | — |
| Kollidon ® VA 64 | — | — | 500 | 500 |
| Polysorbate 80 | — | 100 | — | 100 |
| Water* qs | 15 ml | 5 ml | 15 ml | 5 ml |
| Aqueous phase | 1.2% | 4% | 1.2% | 4% |
| (concentration of CRL 42249 in the | | | | |
| solution) | | | | |
| Lyophilized product (concentration of | 12.5% | 14.2% | 17.6% | 25% |
| CRL 42249 in the dry final product) | | | | |

*The water is eliminated by sublimation during the process

After lyophilization, the products of compositions 11 to 14 are dissolved in water (50 ml) with stirring; the dissolution is verified after 10 minutes of stirring, the absence of solid particles attesting to complete solubilization. For these four formulations, complete dissolution (100% of active agent dissolved) was observed after 10 minutes.

Thus, the oral lyophilized formulations in accordance with the invention made it possible to 100% resolubilize an active ingredient that has, however, particularly low solubility in water. In allowing as complete and as rapid a dissolution as those using β-cyclodextrin, which is a powerful solubilizing agent, the formulations of the invention appear to be a solid and simple alternative for improving the dissolution of very-low-solubility active agents, since the amount of copovidone used is much lower than the amount of β-cyclodextrin. The active ingredient/copovidone ratio in these two formulations is 0.4.

### Example 5

### Formulations Using a Solution of Meloxicam

In this example, three formulations of oral lyophilisates in accordance with the invention, based on meloxicam, copovidone and nonionic surfactant, were prepared in order to improve the dissolution and the bioavailability of this active ingredient.

Meloxicam is an active ingredient of the nonsteroidal anti-inflammatory (NSAI) family which, like most oxicams, is a particularly water-insoluble lipophilic active agent. This characteristic makes the product difficult to formulate in injectable solution form. Moreover, meloxicam is absorbed slowly in the organism, with a Tₘₐₓ of about 5 to 6 hours and a half-life of about 15 to 20 hours. The very low solubility of the active agent limits all the more its absorption.

Owing to this low solubility, the product is indicated only for chronic pathological conditions such as rheumatoid arthritis or osteoarthritis, for example, but the slowness with which the product is absorbed makes it unavailable for treating acute conditions.

Several authors have thus attempted to improve the solubility of meloxicam, most by using microemulsions (Physicochemical Properties and Evaluation of Microemulsion Systems for Transdermal Delivery of Meloxicam; ZHONG DA-Gang et al; Chem. Res. Chinese U. 2007, 23(1), 81-86) or nanoparticulate formulations (Nanoparticulate Meloxicam Formulations; Elan Corporation plc—US 2004 229038). However, no formulation in the form of a true solution of meloxicam has for the moment been prepared.

However, the Applicant has succeeded in producing three compositions in accordance with the invention by going through an aqueous liquid phase in which the meloxicam is completely dissolved. These formulations are obtained by mixing the copovidone (Kollidon® VA 64, BASF), the surfactant (Polysorbate 80), the pH-increasing agent (sodium hydroxide) and, where appropriate, a pH-stabilizing buffering agent ((hydroxymethyl)aminomethane or “Tris buffer”) in purified water with stirring until a clear solution is obtained. The pH stabilizer makes it possible, according to the amount of sodium hydroxide present in the medium, to avoid a variation in pH that could cause the active ingredient to precipitate.

Thus, the following are added in order: first the copovidone, the polysorbate, the sodium hydroxide and, where appropriate, the (hydroxymethyl)aminomethane, and then the meloxicam with stirring at a temperature ranging from 25 to 50° C. until complete dissolution of the meloxicam.

The solution is then cooled to between 20° and 30° C. and mannitol (diluent and filler which gives its structure and solidity to the lyophilisate) is added with stirring until a homogeneous suspension is obtained.

The resulting suspension is then distributed into preformed blisters and then, as is described in Example 1, frozen and lyophilized.

The three formulations in accordance with the invention have the compositions as described in Table 7.

TABLE 7

| | Formulations | | |
|---|---|---|---|
| | 15 | 16 | 17 |
| | (mg/unit) | (mg/unit) | (mg/unit) |
| | batch | batch | batch |
| | 125.07 | 129.07 | 126.07 |
| Meloxicam | 7.5 | 7.5 | 7.5 |
| Copovidone | 100.0 | 100.0 | 100.0 |
| (Kollidon ® VA 64) | | | |
| Polysorbate 80 | 5.0 | 5.0 | 5.0 |
| (Hydroxymethyl)aminomethane | 28.3 | 14.0 | 0 |
| Water | 395.0 | 395.0 | 395.0 |
| 1N NaOH | 20.0 | 25.0 | 55.0 |
| Appearance | Clear | clear | clear |
| | solution | solution | solution |
| | yellow | yellow | yellow |
| Mannitol | 239.2 | 253.5 | 267.5 |
| Appearance | Sus- | Suspension | Suspension |
| | pension | yellow | yellow |
| | yellow | | |
| Weight of the suspension | 795 mg | 800 mg | 800 mg |
| Dry weight of the final product | 380.8 mg | 381 mg | 382.2 mg |
| Characterization of the final | | | |
| product | | | |
| Hardness (in Newtons) | 50-70 N | 30-50 N | 10-20 N |
| Disintegration time | 25-35 sec | 20-30 sec | 10-20 sec |
| pH | 9.3 | 8.9 | 7.5 |

### Example 6

### Formulations Using a Meloxicam Microemulsion

In order to obtain complete dissolution of the meloxicam, a meloxicam formulation (Formulation 18) is prepared in the form of a microemulsion through the use of a cosurfactant (propylene glycol) of two nonionic surfactants based on di- and triglycerides (sold under the respective trademarks Capmul® and Captex).

Two formulations in the form of lyophilized tablets (Formulations 19 and 20) are then prepared from this microemulsion.

### Formulation 18:

All the excipients mentioned in Table 8 are mixed, in a mixer, in the presence of purified water, in the proportions indicated.

TABLE 8

| Formulation 18: Self-emulsifying formulation | (mg/unit) |
|---|---|
| Meloxicam | 7.5 |
| (Hydroxymethyl)aminomethane | 6.7 |
| Propylene glycol | 20.0 |
| Polysorbate 80 | 42.0 |
| Capmul ® MCM (medium-chain mono/diglycerides) | 5.0 |
| Captex ® 355 (caprylic/capric acid triglycerides) | 3.0 |
| Copovidone | 2.0 |
| Purified water | 13.3 |

The meloxicam is then dissolved in this mixture brought to a temperature of 60° C. with stirring until complete dissolution both of the active ingredient and of the excipients.

An emulsion comprising lipid microdroplets made up of the medium-chain mono- and diglycerides and the triglycerides then forms spontaneously during stirring. The meloxicam is in the dissolved state in the lipid microdroplets.

### Formulation 19:

Mannitol, dextran and purified water are added to the microemulsion of Formulation 18, in the proportions specified in Table 9, with stirring until a homogeneous suspension is obtained. This suspension is then, as in Example 1, distributed into preformed blisters and lyophilized.

TABLE 9

| Formulation 19: Lyophilization of a formulation in | (Batch 138.07) |
|---|---|
| microemulsion form | (mg/unit) |
| Composition (according to formulation 18) | 100 mg |
| Mannitol | 260 mg |
| Dextran 70 | 40 mg |
| Purified water | 300 mg |
| Weight of the suspension | 700 mg |
| Weight of the dry final product | 387 mg |
| Characterization of the final product | |
| Hardness | 15-25 N |
| Disintegration rate | 10-15 seconds |
| pH | 8.6 |

Comparative dissolution tests such as those carried out in Example 1 are then carried out on Formulations 15 and 17 (described in Example 5), on Formulation 19 and on a formulation of meloxicam available on the market (Mobic®).

The results of these tests are the following:
Formulation 15 (Batch 125.07): 80% of meloxicam dissolved in 8 minutes.
Formulation 17 (Batch 126.07): 80% of meloxicam dissolved in 10 minutes.
Formulation 19 (Batch 138.07): 80% of meloxicam dissolved in 8 minutes.
Formulation of the prior art (Mobic®): 50% of meloxicam dissolved in 10 minutes.

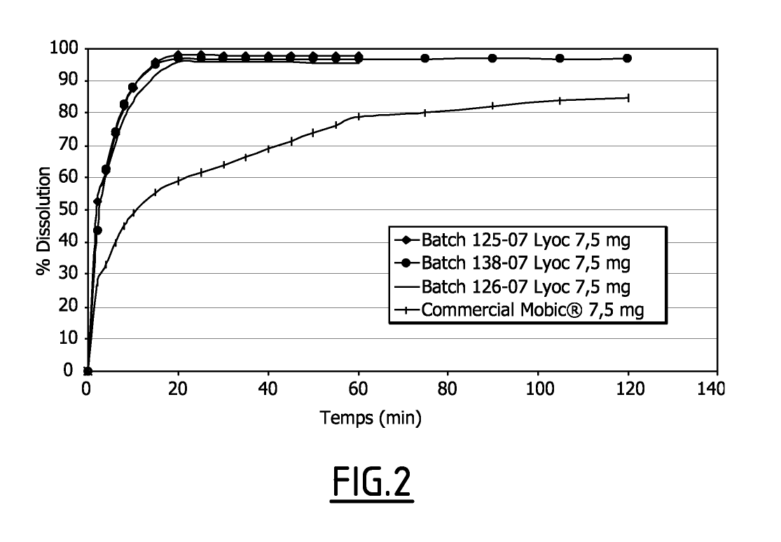

The results of the dissolution tests carried out (reported in FIG. 2) show that, compared with the market formulation, and for the same dosage (7.5 mg of meloxicam), more than 80% of the meloxicam is dissolved in 10 minutes for the formulations according to the invention, whereas this percentage at 10 minutes is only 50% for the tablets according to the prior art.

The same tests are repeated on these formulations after 8 months of storage at ambient temperature and the three formulations according to the invention display the same dissolution profile.

### Example 7

### Pharmacokinetics of Lyophilized Formulations of Meloxicam, in Dogs

In this example, two formulations: Formulation 20 (Batch 001-08) and Formulation 21 (Batch 367-07), in accordance with the invention, of oral lyophilisates of meloxicam, details of the formulae of which are given in Tables 10 and 11 below, were administered as a single dose to conscious dogs with the aim of comparing the rate of absorption of meloxicam when it is formulated in accordance with the invention or in accordance with the product currently on the market (Mobic®), details of the composition of which are given hereinafter.

The meloxicam tablet formulation on the market (Mobic®) contains 7.5 mg of meloxicam comprising the following excipients: sodium citrate, lactose monohydrate, microcrystalline cellulose, povidone, anhydrous colloidal silica, crospovidone, magnesium stearate.

Each of the three formulations was administered to three dogs.

Formulation 20 (Batch 001-08, Table 10) is a formulation in oral lyophilisate form, obtained from a microemulsion according to the invention as described in the previous example (identical to Formulation 19), and Formulation 21 (Batch 367-07, Table 11) is a lyophilized formulation obtained from a solution of copovidone and meloxicam.

Formulation 20 (from a Meloxicam Microemulsion)

TABLE 10

| | Batch number | |
|---|---|---|
| | 001-08 (batch size: 250 | |
| | tablets)/Formulation 20 | |
| | Ingredients | Amount (g) |
| Composition of the formulation | Meloxicam | 15.00 |
| | Tris buffer | 13.33 |
| | Propylene glycol | 40.00 |
| | Capmul ® MCM | 10.00 |
| | Captex ® 355 | 6.00 |
| | Copovidone | 4.00 |
| | (Kollidon VA 64) | |
| | Montanox 80 PHA | 85.00 |
| | Dextran 70 | 80.00 |
| | Sucralose | 20.00 |
| | Cola flavor | 50.00 |
| | Mannitol | 520.00 |
| | Purified water | 626.67 |
| | NaOH (1N) | — |
| | Total dry mass | 843.33 |
| | Wet mass | 1470.00 |

| | |
|---|---|
| Formulation procedures | PVC blister size: 16 × 9 mm |
| | Distribution volume: 1270 μl |
| Tablet characteristics | Hardness: 57.5 N ± 7% |
| | Weight uniformity: 826.9 mg ± 0.73% |
| | Water content: 1.40% |
| | Taste: acceptable |
| | Disintegration (in 50 ml of water): 14 |
| | seconds |

Formulation 21 (from a meloxicam solution)

TABLE 11

| | Batch number | |
|---|---|---|
| | 367-07 (batch size: 250 | |
| | tablets)/Formulation 21 | |
| | Ingredients | Amount (g) |
| Composition of the formulation | Meloxicam | 15.00 |
| | Tris (pH buffer) | 10.00 |
| | Copovidone | 200.00 |
| | (Kollidon ® VA 64) | |
| | Montanox 80 PHA | 10.00 |
| | Sucralose | 20.00 |
| | Cola flavor | 50.00 |
| | Mannitol | 495.00 |
| | Purified water | 600.00 |
| | NaOH | 115.00 |
| | Total dry mass | 800 |
| | Wet mass | 1515 |

| | |
|---|---|
| Formulation procedures | PVC blister size: 16 × 9 mm |
| | Distribution volume: 1330 μl |
| Tablet characteristics | Hardness: 30.6 N ± 15% |
| | Weight uniformity: 818.8 mg ± 1.36% |
| | Water content: 1.74% |
| | Taste: acceptable |
| | Disintegration (in 50 ml of water): 55 |
| | seconds |

The results of this pharmacokinetic study are reported in Table 12 below.

TABLE 12

| | | | Tₘₐₓ (min, | Cₘₐₓ, in |
|---|---|---|---|---|
| | Number | Dose, | max), in | ng/mL |
| Formulations tested | of dogs | in mg | minutes | (±SD) |
| Formulation 21 | 3 | 15 | 60 (15, 120) | 6834 ± 1070 |
| (solution) | | | | |
| (Batch No. 367-07) | | | | |
| Formulation 20 | 3 | 15 | 45 (45, 480) | 7009 ± 1003 |
| (microemulsion) | | | | |
| (batch No. 001-08) | | | | |
| Mobic ® (commercial | 4 | 7.5 | 480 (240, 720) | 3175 ± 566 |
| prior art formulation) | | | | |

The pharmacokinetic profiles obtained show a slow meloxicam elimination phase in accordance with what has been observed, moreover, in the literature. However, the two Formulations (20 and 21) in accordance with the invention demonstrate a rapid absorption of meloxicam compared with that of the product on the market, with a Tₘₐₓ of 45 to 60 minutes for the formulations of the invention compared with a Tₘₐₓ of about 480 minutes for the Mobic® conventional tablet formulation.

### Example 8

### Pharmacokinetics of Lyophilized Formulations of CRL 42249C, in Dogs

A study consisting in studying the plasma kinetics of the CRL 42249C compound was carried out on dogs so as to determine the influence of Kollidon® VA 64 on the absorption of this active ingredient.

A cyclodextrin-based lyophilized preparation and two copovidone-based lyophilized preparations were prepared as in the previous example. The compositions thereof are summarized in Table 13:

TABLE 13
Amounts (in grams)

| | Formulation 23 | Formulation 24 | Formulation 25 |
|---|---|---|---|
| | (ref. 356-96-1) | (ref. 356-96-2) | (ref. 222-96) |
| Active | 2 (17.6%) | 2 (25.2%) | 4 (3.84%) |
| ingredient | | | |
| (CRL-42249C | | | |
| Copovidone | 10 | 5 | — |
| (Kollidon VA | | | |
| 64) | | | |
| Surfactant | — | 1 | — |
| (Polysorbate | | | |
| 80) | | | |
| Hydroxy-beta- | — | — | 100 |
| cyclodextrin | | | |
| Water (*) | 30 | 40 | 300 |

(*) Eliminated during sublimation

The tests carried out consisted of the oral administration of 90 mg/kg of CRL 42249C alone (Formulation 22) or of 10 mg/kg of the CRL 42249C compound either mixed with copovidone (Formulation 23) or mixed with a copovidone/surfactant combination (Formulation 24), or of 10 mg/kg of the CRL 42249C compound mixed with cyclodextrin (Formulation 25).

### Procedure:

Each trial is carried out in two male Beagle dogs. Each dog, after having been given no food for 12 hours, receives, orally, 10 mg/kg (or 90 mg/kg for Formulation 22) of CRL 42249C in the form of three tablets per formulation.

Ten samples per dog are taken at T=0 (control sample); 0.5 h; 1 h; 1.5 h; 2 h; 2.5 h; 3 h; 4 h; 6 h and 8 h after administration.

The CRL 42249C concentrations in the blood are determined after liquid/liquid extraction with 10 ml of ether in a basic medium. The organic phase is evaporated and then taken up with 200 ml of mobile phase, 80 ml of which will be injected into the chromatograph. The separation is then carried out by high performance liquid chromatography (on a Cs Ultrabase KR 135 (No. 36)) using spectrophotometric detection in the ultraviolet range (UV spectrophotometer, Spectro Monitop 3200, LDC analytical). The mobile phase is composed of a mixture of acetonitrile and potassium phosphate adjusted to pH=2.5 with ortho-phosphoric acid.

The mean of the results obtained for each Formulation 22 to 25 is reproduced in Table 14:

TABLE 14

| | Number of | Tₘₐₓ | Cₘₐₓ |
|---|---|---|---|
| | dogs | (hours) | (ng · ml) |
| Formulation 22 | 2 | 3 | 33.5 |
| (active ingredient alone at 90 mg/kg) | | | |
| Formulation 23 | 2 | 2.5 | 184.1 |
| (Copovidone) | | | |
| Formulation 24 | 2 | 1.75 | 103.6 |
| (Copovidone + surfactant) | | | |
| Formulation 25 | 2 | 1.25 | 68.25 |
| (cyclodextrin) | | | |

The two formulations in accordance with the invention improve both the rate of absorption of CRL 42249C and the amount of CRL 42249C absorbed compared with the administration of CRL 42249C alone. Specifically, although the amount of active ingredient administered is 9 times lower for the formulations of the invention than for CRL 42249C administered alone, the Cₘₐₓ values observed are 3 to 5 times higher for the formulation with surfactant and for the formulation with copovidone alone, respectively. The galenical formulations in accordance with the invention therefore make it possible to significantly improve the absorption of this insoluble active ingredient.

Compared with a formulation based on cyclodextrin which serves as an agent for facilitating dissolution (Formulation 25), the two formulations of the invention make it possible to significantly increase the amount of active ingredient absorbed, especially for the formulation based on copovidone alone, for which the amount of active ingredient absorbed is approximately 3 times higher than for the formulation based on cyclodextrin.

Thus, even in the absence of surfactant, the copovidone-based lyophilized formulations of the invention make it possible to effectively improve the absorption of active ingredients with very low solubility in water.

What is claimed is:

1. A pharmaceutical composition in oral lyophilizate form, which is obtained by lyophilization of an aqueous liquid phase free of organic solvent and of ionic surfactant, containing at least one active ingredient which has low solubility or very low solubility or which is virtually insoluble in water, and one polyvinylpyrrolidone/polyvinyl acetate copolymer in a PVP/VA ratio of 60/40.

2. The pharmaceutical composition as claimed in claim 1, wherein the active ingredient/(polyvinylpyrrolidone/polyvinyl acetate copolymer) ratio is between 1:40 and 8:1.

3. The pharmaceutical composition as claimed in claim 1, wherein the active ingredient/(polyvinylpyrrolidone/polyvinyl acetate copolymer) ratio is between 1:20 and 5:1.

4. The pharmaceutical composition as claimed in claim 1, wherein said liquid aqueous phase is a microemulsion.

5. The pharmaceutical composition as claimed in claim 4, wherein said active ingredient is meloxicam.

6. The pharmaceutical composition as claimed in claim 1, which also comprises one or more nonionic surfactants approved for pharmaceutical use.

7. The pharmaceutical composition as claimed in claim 6, wherein the amount of nonionic surfactant approved for pharmaceutical use represents between 0.01% and 20% by weight relative to the dry weight of said composition.

8. The pharmaceutical composition as claimed in claim 1, wherein, when said aqueous liquid phase is a solution, a dispersion or a suspension, the amount of nonionic surfactant approved for pharmaceutical use represents between 0.05% and 0.5% by weight relative to the dry weight of said composition.

9. The pharmaceutical composition as claimed in claim 1, wherein said nonionic surfactants belong to the polysorbate group.

10. The pharmaceutical composition as claimed in claim 1, which also comprises one or more excipients belonging to the group comprising binders, cosurfactants, diluents, gelling agents, viscosity modifiers, fillers, cohesion agents, emulsifiers, sweeteners, colorants, taste-masking agents, flavors, pH-increasing agents and pH stabilizers.

11. The pharmaceutical composition as claimed in claim 1, wherein said active ingredient belongs to the group comprising fenofibrate, meloxicam, methanesulfonate salts of dihydropyridine, hydrochlorides of dihydropyridine and molecules belonging to the dihydropyridine group.

12. An oral lyophilizate comprising at least one polyvinylpyrrolidone/polyvinyl acetate copolymer, said polyvinylpyrrolidone and polyvinyl acetate being in a PVP/VA ratio equal to 60/40, and one active ingredient which has low solubility or very low solubility or which is virtually insoluble in water, wherein the active ingredient/(polyvinylpyrrolidone/polyvinyl acetate copolymer) ratio is between 1:20 and 5:1, provided that if the active ingredient is docetaxel and the copolymer is PVP-VA 64, the active ingredient/copolymer ratio does not equal to 300/2,700 (0.11).

13. An oral lyophilizate according to claim 1 comprising at least one polyvinylpyrrolidone/polyvinyl acetate copolymer, said polyvinylpyrrolidone and polyvinyl acetate being in a PVP/VA ratio equal to 60/40, and one active ingredient which has low solubility or very low solubility or which is virtually insoluble in water, wherein the active ingredient/(polyvinylpyrrolidone/polyvinyl acetate copolymer) ratio is between 1:20 and 5:1.

14. The oral lyophilizate as claimed in claim 12, wherein said active ingredient belongs to the group comprising fenofibrate, meloxicam, methanesulfonate salts of dihydropyridine, hydrochlorides of dihydropyridine and molecules belonging to the dihydropyridine group.

15. A process for producing an oral lyophilizate comprising at least one active ingredient, comprising the steps of:
a) preparation of a homogeneous aqueous microemulsion liquid phase free of ionic surfactant or of organic solvent, comprising said active ingredient(s) in solution, suspension or emulsion and a polyvinylpyrrolidone/polyvinyl acetate copolymer, said polyvinylpyrrolidone and polyvinyl acetate being in a PVP/VA ratio equal to 60/40, dissolved in this aqueous phase,
b) sublimation of the water contained in the liquid phase and, optionally,
c) additional drying of the resulting formulation.

16. The use of a pharmaceutical formulation as claimed in claim 1, for the production of a medicament in the form of a lyophilized tablet that can be administered orally and that comprises at least one active ingredient which has low solubility or very low solubility or which is virtually insoluble in water.
